(19) United States
(12) Patent Application Publication
(10) Pub. No.: US 2010/0166859 A1
(43) Pub. Date: Jul. 1, 2010

(54) NOVEL NUTRACEUTICAL COMPOSITIONS AND USE THEREOF

(76) Inventors: Luppo Edens, Rotterdam (NL); Regina Goralczyk, Grenzach-Wyhlen (DE); Adrian Wyss, Basel (CH)

Correspondence Address: NIXON & VANDERHYE, PC, 901 NORTH GLEBE ROAD, 11TH FLOOR, ARLINGTON, VA, 22203, US

(21) Appl. No.: 12/160,823

(22) PCT Filed: Dec. 29, 2006
(86) PCT No.: PCT/EP06/12595

§ 371 Date: Jul. 23, 2009

(30) Foreign Application Priority Data
Jan. 16, 2006 (EP) 06 000 828.1

(51) Int. Cl. A61K 38/06; C07K 4/00; A61P 25/24; A61P 25/28; A61K 9/28

(52) U.S. Cl. 424/474; 530/331; 514/18

(57) ABSTRACT

The invention relates to a novel nutraceutical composition containing at least one tripeptide as active ingredient. The term “nutraceutical' as used herein denotes a usefulness in both the nutritional and pharmaceutical field of application. Thus, the novel nutraceutical compositions can find use as supplement to food and beverages, and as pharmaceutical formulations for enteral or parenteral application which may be solid formulations such as capsules or tablets, or liquid formulations, such as solutions or suspensions. As will he evident from the foregoing, the term nutraceutical composition also comprises food and beverages containing the above-specified active ingredients, for example protein hydrolysates which are rich in tripeptides according to the invention. The compositions are useful to improve cognitive functions such as learning, memory and alertness and for the treatment of a disorder connected to impaired neurotransmission and of conditions resulting from hypoxia and for alleviating neuropathic pain.

The present invention relates to a novel nutraceutical composition containing at least one tripeptide as active ingredient.

The term “nutraceutical” as used herein denotes a usefulness in both the nutritional and pharmaceutical field of application. Thus, the novel nutraceutical compositions can find use as supplement to food and beverages, and as pharmaceutical formulations for enteral or parenteral application which may be solid formulations such as capsules or tablets, or liquid formulations, such as solutions or suspensions. As will be evident from the foregoing, the term nutraceutical composition also comprises food and beverages containing the above-specified active ingredients, for example protein hydrolysates which are rich in tripeptides according to the invention.

The invention further relates to the use of these tripeptides as food additives or nutraceuticals to improve cognitive functions such as learning, memory and alertness. The tripeptides are also useful for treating conditions resulting from hypoxia and for alleviating neuropathic pain and as mood improver.

Memory, learning and alertness rely on neuronal circuits in the midbrain, especially in hippocampus where information is processed and memory is consolidated. Mental performance and learning is dependent on synaptic plasticity, this means the recruitment of new receptors, formation of new synapses and eventually the generation of new neuronal connections.

L-Glutamate is the major excitatory neurotransmitter in the mammalian CNS, acting through both ligand gated ion channels (ionotropic receptors) and G-protein couplend (metabotrobic) receptors. Activation of these receptors is responsible for basal excitatory synaptic transmission and many forms of synaptic plasticity such as long-term potentiation (LTP) and long-term depression (LTD), which are thought to underlie learning and memory. They are thus also potential targets for therapies for CNS disorders such as epilepsy and Alzheimer's disease, but also for mood disorders such as depression.

The ionotropic receptors themselves are ligand gated ion channels, i.e. upon binding of glutamate that has been released from a presynaptic cell, charged ions such as Na⁺ and Ca²⁺ pass through a channel in the centre of the receptor complex. This flow of ions results in a depolarization of the plasma membrane and the generation of an electrical current that is propagated down the processes (dendrites and axons) of the neuron to the next in line.

The ionotropic glutamate receptors are multimeric assemblies of four or five subunits, and are subdivided into three groups (AMPA, NMDA and Kainate receptors).

NMDA (N-methyl-D-aspartate) receptors are composed of assemblies of NR1 subunits and NR2 subunits. The glutamate binding domain is formed at the junction of NR1 and NR2 subunits. In addition to glutamate, the NMDA receptor requires a co-agonist, glycine, in order to modulate the receptor's function. The glycine binding site is found on the NR1 subunit. The NR2 subunit also possesses a binding site for polyamines, regulatory molecules that modulate the functioning of the NMDA receptor. At resting membrane potentials, NMDA receptors are inactive. This is due to a voltage-dependent block of the channel pore by magnesium ions, preventing ion flows through it. NMDA receptors are permeable to calcium ions as well as being permeable to other ions. Thus NMDA receptor activation leads to a calcium influx into the post-synaptic cells, a signal thought to be crucial for the induction of NMDA-receptor dependent LTP and LTD (animation).

AMPA (alpha-amino-3-hydroxy-5-methyl-4-isoxazole propionate) receptors mediate fast synaptic transmission in the CNS and are composed of four subunits. Native AMPA receptor channels are impermeable to calcium, a function controlled by one subunit.

Both AMPA and NMDA glutamatergic receptor subtypes in hippocampus have been shown to induce LTP, a form of synaptic modification believed to be involved in memory formation. Because of their postsynaptic localization, any differential induction of LTP by the two receptor subtypes would strongly support the existence of a postsynaptic mechanism of LTP activation. (Differential Expression of Short-term Potentiation by AMPA and NMDA Receptors in Dentate Gyrus”, X Xie, G Barrionuevo and T W Berger, Learning & Memory, Vol 3, 115-123, Copyright ©1996 by Cold Spring Harbor Laboratory Press)

LTP and LTD are mediated by the production of brain derived neurotrophic factor (BDNF), which is induced upon stimulation of glutaminergic neurotransmission. Increased expression of brain-derived neurotrophic factor (BDNF) appears also to be involved in the mechanism of action of antidepressant drugs. Therefore it has been proposed that potentiation of the AMPA receptor function may also be useful in the treatment of mood disorders such as depression.

The AMPA receptor has an allosteric modulatory site(s) for which potent positive modulators (potentiators) have been designed. These compounds produce antidepressant-like effects in animal models, increase levels of brain-derived neurotrophic factor (BDNF) and engender neurogenesis in vivo. Although these effects are also produced by traditional antidepressants, AMPA receptor potentiators appear to produce their effects through a novel mechanism.

It is thought that drugs that can modulate or otherwise alter the operation of the NMDA and/or AMPA receptor may improve cognitive functions, i.e. NMDA receptor-associated memory problems, or mood disorders or conditions such as hypoxia (a deficiency of oxygen reaching the tissues of the body). In addition, NMDA receptors are associated with detection of pain. Other drugs that interact with the NMDA receptor may enhance the ability of the cells to form LTP and thus enhance learning and memory.

There is also an increasing interest in the development of compounds as well as nutraceuticals compositions that may be used to improve learning memory and alertness for example in elderly people, students and persons who are preparing exams and people who are under mental stress, and/or that may be used to treat mental diseases/disorders or to prevent the development of mental diseases/disorders such as depression in people at risk, and to stabilize mood.

Now it has been found that tripeptides with one or two C-terminal prolins have the ability to bind to the NMDA and/or AMPA receptor. Upon binding both receptors are activated and lead to a Ca²⁺-influx into the cell. This is the first step to induce transcriptional activation of a number of genes and subsequently to induce LTP, the main cellular mechanism involved in memory formation and memory consolidation.

Therefore one aspect of the invention is a novel nutraceutical composition comprising as active ingredient at least one tripeptide encompassed by the general formula

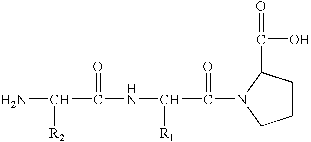

a salt, derivative, metabolite or analogue thereof, wherein
R₁ represents a residue derived from amino acids Gly (G) or Pro (P) and
R₂ indepently represents a residue derived from any of the amino acids Gly (G), Glu (E), Pro (P), Cys (C), Asp (D), Phe (F), His (H), Ser (S), Gln (Q), Arg (R), Thr (T) or
the N-terminal amino acid is Pyroglutamate.

All (oligo)peptide and polypeptide formulas or sequences herein are written from left to right in the direction from amino-terminus to carboxy-terminus, in accordance with common practice. The one-letter code of amino acids used herein is commonly known in the art and can be found in Sambrook, et al. (Molecular Cloning: A Laboratory Manual, 2nd, ed. Cold Spring Harbor Laboratory, Cold Spring Harbor Laboratory Press, Cold Spring Harbor, N.Y., 1989).

Tripeptides with a C-terminal proline are supposed to be stable in the GI tract and in the plasma. They are not degraded or less degraded by human peptidases and they have a longer half life time.

The term derivative, metabolite or analogue thereof as used herein comprises etherified or esterified hydroxy groups which may be derived from unsubstituted or substituted, straight or branched alkyl groups or from unsubstituted or substituted, straight or branched aliphatic, araliphatic or aromatic carboxylic groups. Etherified hydroxy groups may further be glycoside groups and esterified hydroxy groups may further be glucuronide or sulfate groups. For example the derivative can be characterized by an optional replacement of the Pro residue with dehydro-Pro, preferably 3,4-dehydro-Pro ; an optional modification of the carboxyl terminus amide group with a substituent selected from a carboxyl group, an hydroxyalkyl group, preferably a hydroxymethyl group, an alkoxycarbonyl group, or an alkylated carbamyl group; optional modification of the amino terminus heterocyclic group or dehydro-heterocyclic group with a substituent selected from the group of a lower alkyl group, preferably having 1 to 3 carbon atoms, a halogen atom, preferably a fluorine or chlorine atom, a hydroxyl group, preferably a cis-ortrans-4-OH-group, a sulphydryl group, preferably a cis-or trans-4-thio-group; or an alkylamino group or a dialkylamino group, preferably a methyl or ethylamino or a dimethyl or diethylamino group; and/or optional modification of the hydrogen atoms at the nitrogen atoms of the amino acid peptide bonds with a lower alkyl group, preferably having 1 to 3 carbon atoms.

The chemical synthesis of the tripeptide according to the present invention is not particularly limited and can be carried out according to known methods, preferably stereospecific processes of peptide chemistry in which the L- or D-configuration of the respective amino acids or their derivatives is maintained.

A process for making an EPP peptide includes chemical synthesis, enzymatic hydrolysis and fermentation of protein containing solutions, this process is advantageously a chemical synthesis production process, using preferably conventional techniques. Production via chemical synthesis is for example possible using conventional techniques as for instance described in “Peptides: Chemistry and Biology” by N. Sewald and H. D. Jakubke, Eds. Wiley-VCH Verlag GmbH, 2002, Chapter 4. Particular cost-effective methods of chemical peptide synthesis suitable for large-scale production are based on the use of alkylchloroformates or pivaloyl chloride for the activation of the carboxylic group combined with the use of methyl esters for C-terminal protection and benzyloxycarbonyl (Z), Formyl (For) or tert-butyloxycarbonyl (BOC) groups for N-protection.

Preferred examples of the active ingredients are: CPP, DPP, EPP, FPP, GPP, HPP, PPP, SPP, DGP, EGP, FGP, GGP, HGP, IGP, PGP, QGP, RGP, SGP, TGP, VGP, PyroglutamatePP.

Most preferred active ingredients are tripeptides which bind to both glutamate receptors with moderate affinity, as for example EPP, PPP, SPP, DGP, GGP, HGP.

The nutraceutical compositions may comprise a combination of at least two active ingredients as defined above, preferably a combination of two tripeptides, one of which interacts with the glycine site of the NMDA receptor and the other interacts with the AMPA receptor.

Specific combinations of at least two active ingredients in the compositions of the present invention comprise:
GGP and EPP
GGP and DGP and/or EGP and/or FGP and/or HGP and/or IGP and/or PGP and/or QGP and/or RGP and/or SGP and/or TGP and/or VGP and/or DPP and/or FPP and/or GPP and/or HPP and/or PPP and/or SPP
EPP and CPP and/or PPP and/or SPP and/or DGP and/or HGP

In another aspect of the invention, the nutraceutical compositions may comprise a proteinhydrolysate containing as major part at least one active ingredient as defined above. Such compositions may be originated from different protein sources, which are treated with a couple of specific peptidases to generate the protein hydrolysates.

In just another aspect, the invention relates to the use of a tripeptide as defined above for the manufacture of nutraceutical compositions for animals including humans to improve cognitive functions such as learning, memory and alertness and/or for the treatment of a disorder connected to impaired neurotransmission, particularly for the manufacture of an antidepressant and/or a mood improver and/or a stress reliever and/or a condition improver. The tripeptides are also useful for treating conditions resulting from hypoxia and for alleviating neuropathic pain.

Animals in the context of the present invention include humans and encompass mammals, fish and birds. Preferred “animals” are humans, pet animals and farm animals.

Examples for pet animals are dogs, cats, birds, toy fish, guinea pigs, (jack) rabbits, hares and ferrets. Examples for farm animals are fish, pigs, horses, ruminants (cattle, sheep and goat) and poultry.

For animals including humans a suitable daily dosage of a tripeptide for the purposes of the present invention may be within the range from 0.001 mg per kg body weight to about 1000 mg per kg body weight per day. More preferred the nutraceutical compositions of the present invention contain at least one, preferably at least two tripeptides in an amount sufficient to administer to a human adult (weighing about 70 kg) a dosage from about 70 mg/day to about 1000 mg/day, preferably from about 70 mg/day to about 500 mg/day.

In a preferred embodiment of the invention, the composition may contain a proteinhydrolysate. Typically the hydrolysate consists of short peptides with the biggest fraction being tripeptides as defined above. Depending on the protein source the composition of the tripeptides can be varied. The proteinhydrolysate is part of the nutraceutical composition such that a dose of 0.01 g/kg body weight/day −3 g/kg bodyweight/day may be administered to a human adult.

The present invention further discloses a nutraceutical composition comprising 5-20 mg/g of a tripeptide as defined above, preferably EPP and/or GGP (on dry matter and on protein).

The nutraceutical compositions according to the present invention may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellyfying agents, gel forming agents, antioxidants and antimicrobials.

Moreover, a multi-vitamin and mineral supplement may be added to the nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

The nutraceutical compositions according to the present invention may be in any galenic form that is suitable for administrating to the animal body including the human body, especially in any form that is conventional for oral administration, e.g. in solid form such as (additives/supplements for) food or feed, food or feed premix, fortified food or feed, tablets, pills, granules, dragées, capsules, and effervescent formulations such as powders and tablets, or in liquid form such as solutions, emulsions or suspensions as e.g. beverages, pastes and oily suspensions. The pastes may be filled into hard or soft shell capsules, whereby the capsules feature e.g. a matrix of (fish, swine, poultry, cow) gelatin, plant proteins or ligninsulfonate. Examples for other application forms are forms for transdermal, parenteral or injectable administration. The dietary and pharmaceutical compositions may be in the form of controlled (delayed) release formulations.

Examples of food are dairy products. Examples of such products are margarines, spreads, butter or yoghurts or milk-drinks.

Examples for fortified food are cereal bars, bakery items such as cakes and cookies.

Beverages encompass non-alcoholic and alcoholic drinks as well as liquid preparations to be added to drinking water and liquid food. Non-alcoholic drinks are e.g. soft drinks, sport drinks, fruit juices, lemonades, teas and milk based drinks. Liquid food are e.g. soups and dairy products. The nutraceutical composition containing a protein hydrolysate may be added to a soft drink, to an energy bar, to a candy such that an adult takes 0.3 to 4 g of said hydrolysates per daily serving.

If the nutraceutical composition is a pharmaceutical formulation the composition further contain a pharmaceutically acceptable excipient, pharmaceutically acceptable diluents or adjuvants. Standard techniques may be used for their formulation, as e. g. disclosed in Remington's Pharmaceutical Sciences, 20th edition Williams & Wilkins, PA, USA. For oral administration, tablets and capsules are preferably used which contain a suitable binding agent, e. g. gelatine or polyvinyl pyrrolidone, a suitable filler, e. g. lactose or starch, a suitable lubricant, e. g. magnesium stearate, and optionally further additives. Preferred are formulations containing 75 to 225 mg, more preferably 100 to 400 mg, of the tripeptide derivate per administration unit, e. g. per tablet or capsule.

In a preferred aspect of the present invention the compositions may be used as a nutritional supplement, e.g., as food additive for the improvement of cognitive functions such as learning, memory and alertness for example in elderly people, students and persons who are preparing exams. This means that the present invention relates to the use of tripeptides according to the invention and with the definitions of R₁ and R₂ and the preferences as given above as “condition improver”, i.e. as means to reduce irritability and tiredness, to reduce or prevent or alleviate physical and mental fatigue, to favour undisturbed sleep, that is to act against insomnia and sleep disorders and to improve sleep, and to increase energy in more general terms, especially to increase the brain energy production, in diseased or normal healthy individuals. Moreover for cognition improvement in general, and especially for maintenance or improvement of attention and concentration, of the memory and of the capacity for remembering, of the learning ability, of the language processing, of problem solving and of intellectual functioning; for improvement of the short-term memory; for increasing the mental alertness; for enhancing the mental vigilance; for reducing the mental fatigue; for supporting cognitive wellness, for maintaining balanced cognitive function, for the regulation of hunger and satiety as well as for the regulation of motor activity. Moreover, tripeptides according to the invention with the definitions of R₁ and R₂ and the preferences as given above as well as compositions comprising an effective dose of them are useful for the treatment, prevention and the alleviation of stress related symptoms, for the treatment, prevention and alleviation of symptoms related to working overload, exhaustion and/or burn out, for the increase of the resistance or tolerance to stress and/or to favor and facilitate the relaxation in normal healthy individuals i.e. such compositions have an effect as “stress reliever”. To achieve all these improvements, an administration over several days (for example at least 4 or 5 days), and better over several weeks is generally preferred.

In another preferred aspect of the present invention the compositions may be used in the treatment of a disorder connected to impaired neurotransmission, particularly as antidepressant or as mood improver in animals including humans. “Mood improver” means that the mood of a person treated with it is enhanced, that the self esteem is increased and/or that the negative thoughts are reduced. It also means the emotions are balanced and/or that the general well being is improved.

In the context of this invention “treatment” also encompasses co-treatment as well as prevention. “Prevention” can be the prevention of the first occurrence (primary prevention) or the prevention of a reoccurence (secondary prevention).

In the context of this invention the term “disorder” also encompasses diseases.

Antidepressants are medicaments for treating mental, behavioural and emotional/affective, neurotic, neurodegenerative, eating and stress related disorders such as e.g. unipolar depression, bipolar depression, acute depression, chronic depression, subchronic depression, dysthymia, postpartum depression, premenstrual dysphoria/syndrom (PMS), climacteric depressive symptoms, aggression, attention deficit disorders (ADS), social anxiety disorders, seasonal affective disorders, anxiety (disorders), fibromyalgia syndrome, chronic fatigue, sleep disorders (insomnia), post-traumatic stress disorders, panic disorders, obsessive compulsive disorders, restless leg syndrome, nervousness, migraine/primary headaches and pain in general, emesis, bulimia, anorexia nervosa, binge eating disorder, gastrointestinal disorders, burn out syndrome, irritability and tiredness.

Antidepressants can also be used for (the manufacture of compositions for) primary and secondary prevention and/or the treatment of neurocognitive impairment. Furthermore they are also effective in the treatment of depressive symptoms or other symptoms related to disturbed neurotransmission occurring as comorbidity in chronic diseases such as cardiovascular diseases, strokes, cancer, Alzheimer disease, Parkinson disease, and others.

In regard to animals, especially pet animals and farm animals can be in conditions in need of enhanced or improved neurotransmission. Such conditions e.g. occur after capture or transport or with housing, when the animals develop analogous disorders and are distressed or aggressive, or display stereotypic behaviour, or anxiety and obsessive-compulsive behaviour.

The following Examples illustrate the invention further. The Examples are for illustrative purposes only and are not intended, nor should they be construed, as limiting the invention in any manner. Those skilled in the art will appreciate that variations and modifications can be made without violating the spirit or scope of the invention.

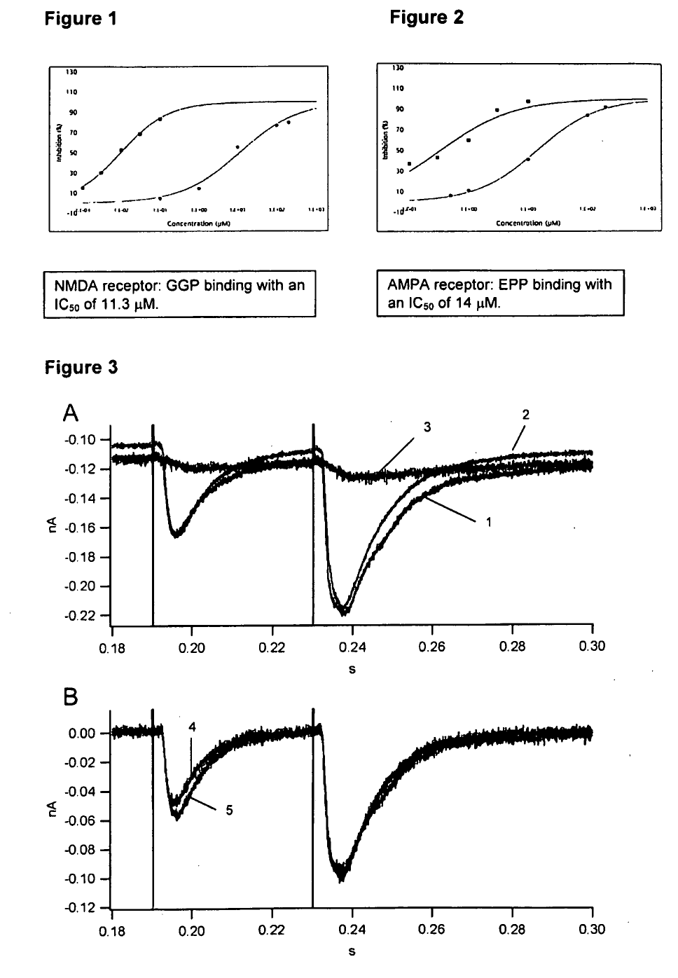

FIG. 1 shows the results of testing the tripeptide GGP as a partial agonist in a pharmacological NMDA-specific binding function measured by a radiolligand binding assay,

FIG. 2 shows the results of testing the tripeptide EPP as a partial agonist in a pharmacological AMPA-specific binding function measured by a radiolligand binding assay,

FIGS. 3A and 3B show the synaptic responses recorded from CA1 pyramidal cells after paired pulse stimulation under baseline condition, with EPP added and with NBQX added, and

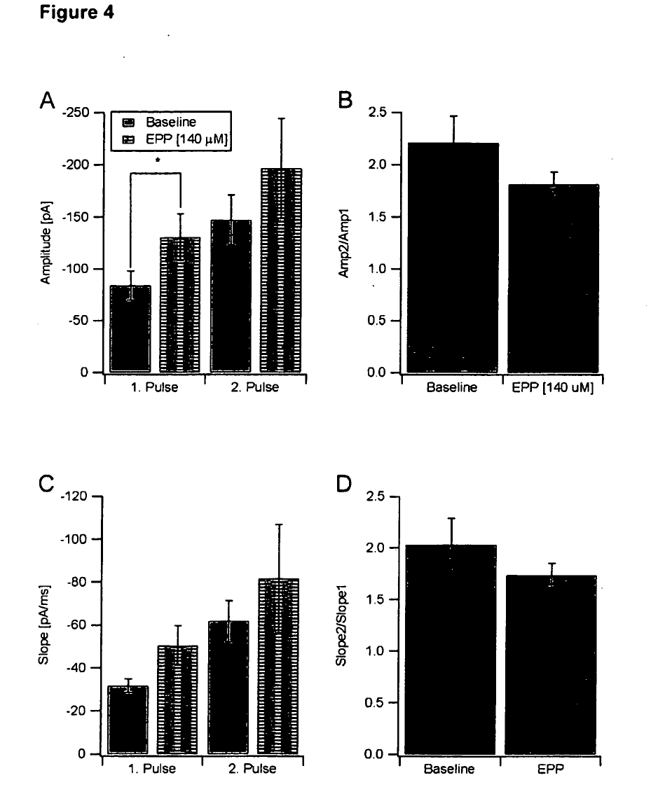

FIGS. 4A-4D show the summary of the synaptic responses recorded from the CA1 pyramidal cells.

### EXAMPLE 1

### NMDA Specific Binding of GGP

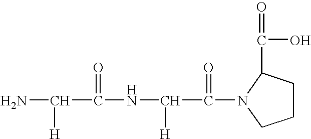

The peptides of the instant invention are capable of specific binding to the mammalian NMDA receptor at the glycine co-agonist site. Because of the significant role the NMDA receptor plays in the mammalian brain, specific agonists are very useful for the fine mapping of NMDA receptor tissue distribution and correlation with disease, injury, or other pharmacological effects. Specific small peptide agonists are particularly useful in that they can be further modified for enhanced bioavailability and for transport across the blood-brain barrier.

Polypeptides or amino acid compositions of the instant invention were tested for the ability to bind to the glycine co-agonist site on the NMDA receptor using a previously validated [3H] MDL-105519 radioligand binding assay (see, for example Siegel B W, Sreekrishna K, Baron B M. Eur J Pharmacol. 1996 Oct. 3; 312(3):357-65). Upon binding, the peptide is displacing the parent radioligand from the NR1a binding site of the NMDA receptor. Binding inhibition of 50% or more is generally considered as significant.

Membrane Preparation

Crude synaptic membranes used in the assay were prepared using rat cerebral cortex tissue (male Wistar rats) and extensively washed using the procedures described previously (Haring et al., 1991, J. Neurochem. 57:323-331). Briefly, tissue which has been stored at −80° C. was homogenized in ice-cold 5 mM Tris (pH 7.4) using a Polytron and then pelleted by centrifugation at 48,000 g for 20 minutes. The resulting supernatant was discarded, and the membranes washed three times in cold buffer. Pellets were then resuspended in 50 mM Tris-acetate (pH 7.4). The membrane suspensions were then pelleted by centrifugation at 48,000 g for 20 minutes and aliquots were stored at −80° C. until use in the assay.

Receptor Binding Assay

Frozen pellets were thawed at room temperature and washed three times by resuspension in 50 mM Tris-acetate (pH 7.4) and centrifugation. Final pellets were suspended at concentrations of 2 to 3 mg/ml in 50 mM HEPES (pH 7.7). Binding reactions were initiated by the addition of 200 μg of freshly prepared membranes to reaction mixtures (about 1 ml final volume) containing 0.33 nM [3H] MDL-105519 at 4° C. in the presence of a range of peptide concentrations (0.5 μM, 1 μM, 10 μM, 100 μM and 200 μM). Incubation time was 30 min. Non-specific binding was determined using 10 μM unlabelled MDL-105519.

Binding reactions were terminated by filtration through a Brandel 24-well cell harvester onto Whatman GFTB glass filters that have been presoaked in 0.25% polyethyleneimine for 30 minutes.

Data Analysis/Interpretation

Assay results are presented as the % inhibition of specific binding. Peptides which competed for [3H] MDL-105519 binding and showed inhibition of at least 50%, were chosen for IC₅₀ determination. Semi-quantitative follow-up to estimate IC₅₀ was done in duplicates at the indicated peptide concentrations.

FIG. 1 shows the binding of GGP in comparison with MDL-105519. FIG. 1 clearly demonstrates dose dependent GGP binding to the glycine site of the NMDA receptor. The GGP tripeptide shows an IC₅₀ of 11.3 μM. The reference compound has an IC₅₀ of 9.6 nM.

### EXAMPLE 2

### AMPA Specific Binding of EPP

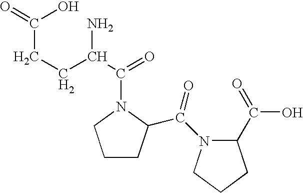

The peptides of the instant invention are capable of specific binding to the mammalian AMPA receptor at the glutamate binding site. Because of the central role of the AMPA receptor in the mammalian brain, specific agonists are very useful for the fine mapping of AMPA receptor tissue expression and distribution and correlation with disease, injury, or other pharmacological effects. Specific small peptide agonists are particularly useful in that they can be further modified for enhanced bioavailability and for transport across the blood-brain barrier.

Polypeptides or amino acid compositions of the instant invention were tested for the ability to bind to the AMPA receptor using a previously validated [3H] AMPA radioligand to binding assay (see, for example Olsen R W, Szamraj O, Houser C R Brain Res. 1987; 402(2):243-54.). Upon binding, the peptide is displacing the parent radioligand from the glutamate binding site of the AMPA receptor. Binding inhibition of 50% or more is generally considered as significant.

Membrane Preparation

The membrane has been prepared as described in example 1.

Receptor Binding Assay

Frozen pellets were thawed at room temperature and washed three times by resuspension in 50 mM Tris-acetate (pH 7.4) and centrifugation. Final pellets were suspended at concentrations of 2 to 3 mg/ml in 50 mM Tris-HCl (pH 7.4, 200 mM KSCN). Binding reactions were initiated by the addition of 200 μg of freshly prepared membranes to reaction mixtures (about 1 ml final volume) containing 5 nM [3H] AMPA at 25° C. in the presence of a range of peptide concentrations (0.5 μM, 1 μM, 10 μM, 100 μM and 200 μM). Incubation time was 90 min. Non-specific binding was determined using 1000 μM unlabelled L-Glutamic acid.

Binding reactions were terminated by filtration through a Brandel 24-well cell harvester onto Whatman GF/B glass filters that have been presoaked in 0.25% polyethyleneimine for 30 minutes.

Data Analysis/Interpretation

Assay results are presented as the % inhibition of specific binding. Peptides which competed for [3H] AMPA binding and showed inhibition of at least 50%, were chosen for IC₅₀ determination. Semi-quantitative follow-up to estimate IC₅₀ was done in duplicates at the indicated peptide concentrations.

FIG. 2: Radioligand binding assay with the AMPA receptor: The FIG. 2 shows a dose dependent EPP binding to AMPA receptor. The EPP tripeptide shows an IC₅₀ of 14 μM. The reference compound has an IC₅₀ of 0.34 μM.

### EXAMPLE 3

### EPP is an Agonistic Modulator of the AMPA Receptor

In order to evaluate the physiological role of EPP at the AMPA receptor, electrophysiology was performed on hippocampal cells.

Experimental Design:

Whole cell patch-clamp experiments were performed in parasagital brain slices on CAI pyramidal cells. The animals used for this study were C57BL/6 mice. After decapitation of the mice brains were quickly removed and placed in aerated (95% O₂, 5% CO₂) artificial cerebrospinal fluid (ASCF) solution at 4° C. The ACSF solution was composed of (mM): 124 NaCl, 2.5 KCl, 1.3 MgCl₂, 26 NaHCO₃, 1.25 NaH₂PO₄, 2.5 CaCl₂ an Synaptic AMPA responses were evoked every 10 seconds with a bipolar platinum electrode and recorded in the voltage clamp configuration with 4-5 MΩ patch pipette at a holding potential of −60 mV. Baseline recordings were performed with 100 μM picrotoxin in the ACSF. After establishing a stable synaptic AMPA response the tripeptide EPP (140 μM) was added to the bath, continuing the recording for an additional 20 to 30 minutes. At the end of every experiment NBQX (20 μM) was bath applied to block the AMPA response. The residual trace was subtracted from the previous recordings to show only the AMPA current. To evaluate possible presynaptic effects of EPP a set of experiments was performed using a paired pulse stimulation protocol with an interpulse interval (IPI) of 40 ms. Averaged synaptic responses from the example experiment are depicted in FIG. 3A and with the subtraction of the non-AMPA response in FIG. 3B.

FIG. 3A: Averaged synaptic response recorded from a CA1 pyramidal cell after paired pulse stimulation (IPI=40 ms) under baseline condition (1), with 140 μM EPP added (2) and with 20 μM NBQX added (3).

FIG. 3B: Same traces as in ‘A’ but with the blue trace subtracted from the baseline (4) and the EPP (5) trace.

Parameters measured to estimate the action of EPP were the holding current, amplitude, and initial slope of the AMPA responses. Additionally the amplitude ratio and initial slope ratio of the two AMPA responses from the paired pulse experiments was determined. The amplitude of the first synaptic AMPA response changed significantly from −83.9±14.1 pA under baseline conditions to −130.6±22.6 pA (p<0.05). The change in slope just failed to reach significance (p=0.06).

Summary of Results

The summary of the results is shown in the FIGS. 4A-4D.

A: Amplitude of the AMPA response after the first and second stimulus (paired pulse protocol) before and after the application of 140 μM EPP.

B: Ratio of the two amplitudes before and after the application of 140 μM EPP.

C: Initial slope of the AMPA response after the first and second stimulus (paired pulse protocol) before and after the application of 140 μM EPP.

D: Ratio of the two slopes before and after the application of 140 μM EPP.

### EXAMPLE 5

### Preparation of a Soft Gelatin Capsule

A soft gelatin capsule (500 mg) may be prepared comprising the following ingredients:

| Ingredient | Amount per Capsule |
|---|---|
| EPP | 200 mg |
| Lecithin | 50 mg |
| Soy bean oil | 250 mg |

Two capsules per day for 3 months may be administered to a human adult for the treatment of mild chronic dysthymia.

### EXAMPLE 6

### Preparation of a Soft Gelatin Capsule

A soft gelatin capsule (600 mg) may be prepared comprising the following ingredients:

| Ingredient | Amount per Capsule |
|---|---|
| EPP | 200 mg |
| Evening prim rose oil | 300 mg |
| Vitamin B₆ | 100 mg |

One capsule per day preferably at the second half of the menstrual cycle may be taken for 14 days for the treatment of premenstrual syndrome and premenstrual dysphoric disorder.

### EXAMPLE 7

### Preparation of a Tablet

A 400 mg-tablet may be prepared comprising the following ingredients:

| Ingredient | Amount per tablet |
|---|---|
| EPP | 100 mg |
| Passion flower standardized extract | 150 mg |
| Green Tea Extract, e.g. TEAVIGO ® | 150 mg |
| from DSM Nutritional Products, | |
| Kaiseraugst, Switzerland | |

For general well being, energizing, to improve learning, memory and alertness for example in elderly people, students and persons who are preparing exams and stress alleviation, one tablet may be taken twice daily for 3 months.

### EXAMPLE 8

### Preparation of an Instant Flavoured Soft Drink

| Ingredient | Amount [g] |
|---|---|
| EPP | 0.9 |
| Sucrose, fine powder | 922.7 |
| Ascorbic acid, fine powder | 2.0 |
| Citric acid anhydrous powder | 55.0 |
| Lemon flavour | 8.0 |
| Trisodium citrate anhydrous powder | 6.0 |
| Tricalciumphosphate | 5.0 |
| β-Carotene 1% CWS from DNP AG, | 0.4 |
| Kaiseraugst, Switzerland | |
| Total amount | 1000 |

All ingredients are blended and sieved through a 500 μm sieve. The resulting powder is put in an appropriate container and mixed on a turbular blender for at least 20 minutes. For preparing the drink, 125 g of the obtained mixed powder are taken and filled up with water to one liter of beverage.

The ready-to-drink soft drink contains ca. 30 mg EPP per serving (250 ml). As a strenghtener and for general well being 2 servings per day (240 ml) may be drunk.

### EXAMPLE 9

### Preparation of a Fortified Non Baked Cereal Bar

| Ingredient | Amount [g] |
|---|---|
| EPP | 0.95 |
| Sugar | 114.55 |
| Water | 54.0 |
| Salt | 1.5 |
| Glucose syrup | 130.0 |
| Invert sugar syrup | 95.0 |
| Sorbitol Syrup | 35.0 |
| Palmkernel fat | 60.0 |
| Baking fat | 40.0 |
| Lecithin | 1.5 |
| Hardenend palm-oil | 2.5 |
| Dried and cut apple | 63.0 |
| Cornflakes | 100.0 |
| Rice crispies | 120.0 |
| Wheat crispies | 90.0 |
| Roasted hazelnut | 40.0 |
| Skim milk powder | 45.0 |
| Apple flavour 74863-33 | 2.0 |
| Citric acid | 5.0 |
| Total amount | 1000 |

EPP is premixed with skim milk powder and placed in a planetary bowl mixer. Cornflakes and rice crispies are added and the total is mixed gently. Then the dried and cut apples are added. In a first cooking pot sugar, water and salt are mixed in the amounts given above (solution 1). In a second cooking pot glucose, invert and sorbitol syrup are mixed in the amounts given above (solution 2). A mixture of baking fat, palmkernel fat, lecithin and emulsifier is the fat phase. Solution 1 is heated to 110° C. Solution 2 is heated to 113° C. and then cooled in a cold water bath. Afterwards solution 1 and 2 are combined. The fat phase is melted at 75° C. in a water bath. The fat phase is added to the combined mixture of solution 1 and 2. Apple flavour and citric acid are added to the liquid sugar-fat mix. The liquid mass is added to the dry ingredients and mixed well in the planetary bowl mixer. The mass is put on a marble plate and rolled to the desired thickness. The mass is cooled down to room temperature and cut into pieces. The non baked cereal bar contains ca. 25 mg EPP per serving (30 g). For general well-being and energizing 1-2 cereal bars may be eaten per day.

### EXAMPLE 10

### Preparation of Protein Hydrolysates Witch are Rich in Tripeptides According to the Invention and Which can be Used in Formulations as Described in Examples 5-9

Background:

A preferred process in which for example the peptides EPP and GGP are generated in high yields comprises the use of a proteolytic enzyme or protease, which cleaves at the carboxy terminus of proline, preferably a proline specific endoprotease or a proline specific oligopeptidase, more preferably a proline specific endoprotease, in combination with a suitable aminopeptidase.

The proline specific endoprotease is preferably capable of hydrolyzing large protein molecules like polypeptides or the protein itself. The process has in general an incubation time of less than 24 hours, preferably the incubation time is less than 10 hours and more preferably less than 4 hours. The incubation temperature is in general higher than 30° C., preferably higher than 40° C. and more preferably higher than 50° C.

High amounts of for example EPP or GGP can be obtained preferably at a degree of hydrolysis (DH) of between 10 and 38, more preferably at a DH of between 15 and 35 and most preferably at a DH of between 20 and 30. The protein used in the present process is preferably a milk protein, more preferably casein or a caseinate. Advantageously the milk protein is not fermented before it is used in the present process. Preferably the insoluble part of the hydrolysed protein is separated from the soluble part under selected pH conditions, preferably acid pH conditions, more preferably at pH between 3.5 and 6 and most preferably at pH between 4 and 5 to result in the composition comprising soluble peptides. In another embodiment of the present invention the amino peptidase is only added after the separation of the insoluble part of the hydrolysed protein.

WO-A 02/45524 describes a proline specific protease obtainable from Aspergillus niger. The A. niger derived enzyme cleaves preferentially at the carboxyterminus of proline, but can also cleave at the carboxyterminus of hydroxyproline and, be it with a lower efficiency, at the carboxyterminus of alanine. WO 02/45524 also teaches that there exists no clear homology between this A. niger derived enzyme and the known prolyl oligopeptidases from other microbial or mammelian sources. In contrast with known prolyl oligopeptidases, the A. niger enzyme has an acid pH optimum. Although the known prolyl oligopeptidases as well as the A. niger derived enzyme are socalled serine proteases, the A. niger enzyme belongs to a completely different subfamily. The secreted A. niger enzyme appears to be a member of family S28 of serine peptidases rather than the S9 family into which most cytosolic prolyl oligopeptidases have been grouped (Rawlings, N. D. and Barrett, A. J.; Biochim. Biophys. Acta 1298 (1996) 1-3). The Aspergillus enzyme is not an oligopeptidase but a true endopeptidase able to hydrolyse intact proteins, large peptides as well as smaller peptide molecules without the need of an accessory endoprotease. This allows us to omit the use of an accessory endoprotease so that hydrolysates with unprecedented high contents of peptides with a carboxyterminal proline residue can be to generated. Furthermore the omission of an accessory endoprotease is preferred to achieve an efficient removal of the bulk of the substrate protein. As a result highly concentrated peptide mixtures characterized by very high proline contents are obtained. Such new hydrolysates can be prepared from different proteinaceous starting materials be it from vegetable or from animal origin. Examples of such starting materials are whey proteins, whey beta-lactoglobulin, whey alpha lactalbumin, caseins, isolated casein fractions, gelatin, fish or egg proteins, potato protein, wheat and maize gluten, soy and pea protein, rice protein as well as lupin protein. From a substrate like gelatin with a high content of proline as well as hydroyproline, hydrolysates with unprecedented high contents of peptides with either a carboxyterminal proline or hydroxyproline residue can be generated. As the A. niger enzyme (like the known prolyl oligopeptidases) enzyme is unable to cleave Pro-Pro or Pro-Hyp, Hyp-Pro or Hyp-Hyp bonds, the approach will also yield hydrolysates containing unprecedented high contents of peptides having two, three or even more carboxyterminal proline or hydroxyproline residues. Obviously the nature and the proline content of the proteinaceous starting material dictates the probability of generating such peptides. Preferred substrates are substrates containing more than 6% proline (i.e. more than 6 grams of this amino acid per 100 gram of protein) such as casein, gelatin, wheat and maize glutens. More preferred substrates are protein fractions from baker's yeast (Saccharomyces cerevisiae), from mouse ear cress (Arabidopsis thaliana), from field mustard (Brassica campestris) and alfalfa (Medicago sativa), the kappa-casein of camel milk and deamidated wheat gluten or deamidated low-molecular-weight glutenin chain of wheat.

Protein substrates containing PEPP or AEPP amino acid sequences are preferred. As the proline-specific endoprotease will cleave carboxyterminal of P, A and PP residues, incubating protein substrates containing PEPP or AEPP amino acid sequences with the proline-specific endoprotease will yield the EPP tripeptide in a single enzyme incubation step. In such a step preferably at least 20%, more preferably at least 30%, most preferably at least 40% of an AEPP or a PEPP sequence present in a protein is converted into EPP. In this approach the EPP content can be enriched by selectively precipitating larger peptides, e.g. by an acid precipitation step.

In view of the fact that peptides carrying such carboxyterminal amino acid sequences can be expected to have a fair chance of surviving the proteolytic activity in the gastrointestinal tract, hydrolysates created by incubation with the A. niger derived prolyl endoprotease provide an excellent starting material for the isolation of known biologically active peptides as well as for the identification of new biologically active peptides.

The hydrolyzing process hinges on the activity of a proline specific endo activity, be it a proline specific oligopeptidase with a neutral pH optimum or a proline specific endoprotease with an acid pH optimum, in combination with an aminopeptidase activity. This incubation with an aminopeptidase can be done at the same time or subsequent to the incubation with the endoprotease. When the different enzymes are incubated simultaneously, conditions have to be choosen so that both enzymes are active, for example at a pH of 6. Commercial enzyme preparations exhibiting aminopeptidolytic activities are Flavourzyme 1000L (Novozymes), Sumizyme FP (Shin Nihon) and Corolase LAP (AB Enzymes). Both Flavourzyme and Sumizyme FP are known to be complex enzyme preparations that contain several aminopeptidolytic enzyme activities besides non-specified endoproteolytic and carboxypeptidolytic activities. Corolase LAP represents a relatively pure, cloned and overexpressed leucine aminopeptidase activity from Aspergillus. Preferably a cloned and overexpressed aminopeptidase with a relatively broad specificity is used to prepare the hydrolysates according to the invention. An example of such an overexpressed enzyme with a relatively broad specificity is aminopeptidase from A. niger (SEQ ID 171 of WO 02/068623).

Aqueous solutions containing protein are highly susceptible for microbial infections, especially if kept for many hours at pH values above 5.0 and at temperatures of 50 degrees C. or below. Especially microbial toxins that can be produced during such prolonged incubation steps and are likely to survive subsequent heating steps and form a potential threat to food grade processes. Unlike the conditions described in EP 1 231 279 the process preferably uses an incubation temperature above 50 degrees C. In combination with the one-step enzyme process in which the enzyme incubation is carried out for a period less than 24 hours, preferably less than 8 hours, more preferably less than 4 hours, the process offers the advantage of an improved microbiological stability.

Preparation of a Protein Hydrolysate which is Rich in EPP:

Camel milk casein incorporates a number of different proteins including beta-casein and kappa-casein. According to the known amino acid sequences the kappa-casein encompasses EPP. More specifically, the EPP sequence is contained in the water soluble part of the kappa-caseine. This water-soluble part, corresponding with the to glycomacropeptide of bovine kappa caseine, is a preferred substrate for EPP isolation.

Hereinabove is described that preferably an -A- or -P-residue is present in the amino acid sequence of the protein N-terminal of (preceding) an EPP sequence. According to the present process the -A- or -P-residue and the E residue of the EPP sequence, preferably 0 to 5 amino acid residues may be present. Upon hydrolysis, the protein will be cleaved C-terminal of the -A- or -P-residue as well as C-terminal of the -P-P-sequence by the proline specific protease. Subsequently the amino acids preceding the EPP sequence will be removed one by one by the aminopeptidase. Because the aminopeptidase is unable to cleave an E-P peptide bond, the EPP sequences will remain intact.

For example in the kappa casein molecule as present in camel milk, the EPP sequence is present in an -ATVEPP-sequence. First the sequence will be cut after -A- and -P-P- by the proline specific endo protease, and subsequently the threonine (T) and the valine (V) residues will be removed by the amino peptidase to result in EPP.

After decantation, filtration or low speed centrifugation to remove the precipitate formed during either the acidification or the solvent addition step, the supernatants containing the biologically active peptides can be recovered. A subsequent evaporation, optionally in combination with an additional filtration step followed by a spray drying step will yield an economical route for obtaining a food grade paste or powder with a high bio-activity and a good water solubility. According to one embodiment of the invention these soluble peptides can be hydrolyzed with an amino peptidase to produce EPP containing hydrolysates. Upon the digestion of caseinates by a proline specific protease in combination with an aminopeptidase, a white and odourless powder with a high concentration of ACE inhibiting peptides, which is rich in EPP is obtained. The preparation as obtained has a very high proline content. The very high proline content of this preparation is unexpected as proline is one of the most hydrophobic amino acids.

If appropriately diluted to the right tripeptide concentration, a versatile starting material with an excellent palatability is obtained suitable for endowing all kinds of foods and beverages. If required, the concentration of the bioactive ingredients can be further increased by subsequent purification in which use is made of the very hydrophobic character of the peptide EPP. Preferred purification methods include nanofiltration, extraction for example with butanol followed by evaporation/precipitation or contacting the acidified hydrolysate as obtained with binders like active carbon or chromatographic resins from the Amberlite XAD range (Rohm). Also butyl-sepharose resins as supplied by Pharmacia can be used. Desorption of the ACE inhibiting peptides from such materials can be done with organic solvents like methanol/ethanol mixtures or with propanol. Furthermore supercritical extraction using CO2 or N2O can be used to obtain highly purified bioactive peptides.

Tripeptides as obtained either before or after an additional (for example chromatography) purification step may be used for the incorporation into nutraceutical compositions as defined above. Preferred serving sizes for the food or beverage or dietary supplements are for example 5-350 grams per serving, for example from 5 to 150 grams. Preferably the number of servings per day is 1-10, for example 2 to 5.

1. Nutraceutical composition comprising as the active ingredient at least one tripeptide encompassed by the general formula 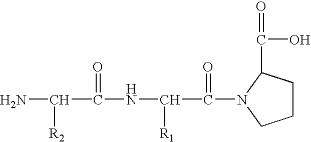
a salt, derivative, metabolite or analogue thereof, wherein
R₁ represents a residue derived from amino acids Gly (G) or Pro (P) and
R₂ independently represents a residue derived from any of the amino acids Gly (G), Glu (E), Pro (P), Cys (C), Asp (D), Phe (F), His (H), Ser (S), Gln (Q), Arg (R), Thr (T), or
the N-terminal amino acid is Pyroglutamate.

2. Composition according to claim 1 containing at least one, preferably two of the following tripeptides: EPP, PPP, SPP, DGP, GGP, HGP, PyroglutamatePP which interacts with the glycine site of the NMDA receptor and/or the with the AMPA receptor.

3. Composition according to claim 1 containing at least GGP and EPP as active ingredients.

4. Composition according to claim 1 containing as active ingredients at least one of the following combinations:
GGP and DGP and/or EGP and/or FGP and/or HGP and/or IGP and/or PGP and/or QGP and/or RGP and/or SGP and/or TGP and/or VGP and/or DPP and/or FPP and/or GPP and/or HPP and/or PPP and/or SPP; EPP and CPP and/or PPP and/or SPP and/or DGP and/or HGP.

5. Composition according to claim 1, which comprises a proteinhydrolysate containing as major fraction at least one active ingredient as defined above.

6. Composition according to claim 1, containing the at least one, preferably at least two tripeptides in an amount sufficient to administer to a human adult (weighing about 70 kg) a dosage from about 70 mg/day to about 1000 mg/day, preferably from about 70 mg/day to about 500 mg/day.

7. Composition according to claim 1 being in form of food such as dairy products (yoghurts), in form of fortified food such as cereal bars and bakery items such as cakes and cookies, in form of dietary supplements such as tablets, pills, granules, dragées, capsules, and effervescent formulations, in form of non-alcoholic drinks such as soft drinks, sport drinks, fruit juices, lemonades, teas and milk based drinks, in form of liquid food such as soups and dairy products.

8. The use of a at least one tripeptide encompassed by the general formula 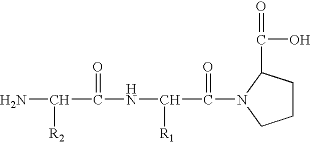
a salt, derivative, metabolite or analogue thereof, wherein
R₁ represents a residue derived from amino acids Gly (G) or Pro (P) and
R₂ indepently represents a residue derived from any of the amino acids Gly (G), Glu (E), Pro (P), Cys (C), Asp (D), Phe (F), His (H), Ser (S), Gln (Q), Arg (R), Thr (T); or
the N-terminal amino acid is Pyroglutamate,
in the manufacture of a nutraceutical composition.

9. The use as in claim 8 wherein the nutraceutical composition is a composition containing at least one, preferably two of the following tripeptides: EPP, PPP, SPP, DGP, GGP, HGP, PyroglutamatePP which interacts with the glycine site of the NMDA receptor and/or the with the AMPA receptor.

10. The use as in claim 8 wherein the nutraceutical composition is intended to improve cognitive functions such as learning, memory and alertness.

11. The use as in claim 8 for the manufacture of a composition for the treatment of a disorder connected to impaired neurotransmission.

12. The use according to claim 11, wherein the composition is an antidepressant, a mood improver, a stress reliever, a condition improver.

13. The use as in claim 8 wherein the nutraceutical composition is a pharmaceutical composition.

14. A method to improve cognitive functions such as learning, memory and alertness in an animal including human, comprising administering an effective dose of at least one tripeptide or salt thereof having a general formula. 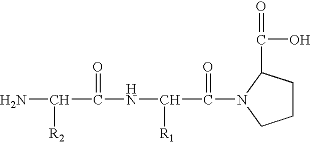
wherein
R₁ represents a residue derived from amino acids Gly (G) or Pro (P) and
R₂ indepently represents a residue derived from any of the amino acids Gly (G), Glu (E), Pro (P), Cys (C), Asp (D), Phe (F), His (H), Leu (I), Ser (S), Gin (Q), Arg (R), Thr (T), Val (V) or
the N-terminal amino acid is Pyroglutamate.

15. A method for the treatment of a disorder connected to impaired neurotransmission in animals including humans, said method comprising administering to the patient an effective dose of at least one tripeptide or salt thereof having a general formula. 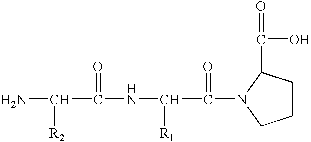
wherein
R₁ represents a residue derived from amino acids Gly (G) or Pro (P) and
R₂ indepently represents a residue derived from any of the amino acids Gly (G), Glu (E), Pro (P), Cys (C), Asp (D), Phe (F), His (H), Leu (I), Ser (S), Gln (Q), Arg (R), Thr (T), Val (V) or
the N-terminal amino acid is Pyroglutamate.

16. The method of claim 14, wherein the tripeptide is selected from the group consisting of EPP, PPP, SPP, DGP, GGP, HGP, PyroglutamatePP.
